(19) United States
(12) Patent Application Publication
(10) Pub. No.: US 2026/0209274 A1
(43) Pub. Date: Jul. 23, 2026

(54) COMPOSITION FOR SKIN REPIGMENTATION

(71) Applicants: Matteo BORDIGNON, Padova (IT); BELLA AURORA LABS, S.A., Barcelona (ES)

(72) Inventors: Matteo BORDIGNON, Padova (IT); Sergi HERNANDEZ NAVARRO, Barcelona (ES); Jardi SEGURA TEJEDOR, Barcelona (ES)

(21) Appl. No.: 19/137,523

(22) PCT Filed: Dec. 13, 2023
(86) PCT No.: PCT/EP2023/085512

§ 371 Date: Jun. 10, 2025

(30) Foreign Application Priority Data
Dec. 16, 2022 (EP) 22383227.0

(51) Int. Cl. C07K 7/08; A61K 9/06; A61K 38/00; A61P 17/00; A61Q 19/00; C07K 5/11; C07K 7/06

(52) U.S. Cl. CPC C07K 7/08; A61K 9/06; A61P 17/00; A61Q 19/00; C07K 5/1019; C07K 7/06; A61K 38/00

(57) ABSTRACT

The present invention relates to a composition for skin repigmentation comprising a peptide inhibitor of MIA (melanoma inhibitory activity) protein, at least an additional peptide, and a pharmaceutically acceptable excipient. The invention also relates to the use of the composition for the prevention and/or treatment of vitiligo. The invention also relates to the cosmetic use of the composition for the repigmentation of skin.

### TECHNICAL FIELD

The present invention relates to the field of skin repigmentation.

### STATE OF THE ART

Depigmentation of skin is a common condition, which can affect different parts of body skin, in different degrees, and can be caused by a number of local and systemic situations.

Usually, depigmentation of the skin is linked to people with vitiligo. Vitiligo is a depigmenting skin disorder, characterized by the selective loss of melanocytes, which in turn results in the disappearance of pigmentation in the affected areas of the skin.

Vitiligo has an estimated prevalence of 0.5-2% of the population in both adults and children, worldwide. Vitiligo is considered as a disease, which should not be dismissed, and whose effects can be psychologically devastating, often with a considerable burden on daily life (Bergqvist C et al., Vitiligo: A Review, Dermatology 2020, 236, 571-592).

Non-segmental vitiligo (NSV) is the most common clinical form of the disease, wherein the patches of depigmentation are usually bilateral and with some form of symmetry.

Vitiligo most commonly involves the face, digits, dorsal hands, flexor wrists, elbows, knees, shins, dorsal ankles, armpits, inguinal area, anogenital area, umbilicus, and nipples.

Vitiligo is characterized by the loss of functional melanocytes, and multiple mechanisms might contribute to this loss, including metabolic abnormalities, oxidative stress, generation of inflammatory mediators, cell detachment and autoimmune responses (Picardo et al., Vitiligo, Nat. Rev. Dis. Primers., 2015, 1, 15011).

The common therapies available at present for the management of vitiligo include topical corticosteroids, topical calcineurin inhibitors, such as tacrolimus and pimecrolimus, photochemotherapy with psoralen plus UVA radiation or with khellin plus UVA radiation, narrowband UVB radiation, oral steroids, immunosuppressants, antioxidants, surgical melanocyte transplantation, or depigmentation of remaining pigmented islands, among others (Taieb A, et al, Guidelines for the management of vitiligo: the European Dermatology Forum consensus, Br. J. Dermatol., 2013, 168 (1), 5-19).

The protein MIA (“Melanoma inhibitory activity”) has been suggested as another target for vitiligo therapy. In particular, in the international patent application WO-A-2012/127352 a number of peptides and peptide derivatives which are MIA inhibitors are proposed for the prevention and treatment of vitiligo.

More recently, in the international patent application WO-A-2022/008251, it has been shown that the combination of the MIA inhibitor peptides disclosed in WO-A-2012/127352 with snail secretion provides a more effective treatment of vitiligo.

Despite the proposals available in the prior art, there is still the need for more therapeutic alternatives for the effective repigmentation of skin.

### OBJECT OF THE INVENTION

The object of the present invention is a composition comprising a MIA inhibitor peptide and at least another peptide.

Another aspect of the invention is said composition for use in the treatment of vitiligo.

Another aspect of the invention is a cosmetic, non-therapeutic use of said composition for repigmenting the skin.

### DESCRIPTION OF THE DRAWINGS

FIG. 1 shows the results of the in vitro repigmentation assay in reconstructed human pigmented epidermis (RHPE) described in Example 1. In ordinates, the increase of the amount of melanin generated with each tested treatment versus the control is represented. In abscises, the grey columns show the results of the assay for the samples treated with the MIA inhibitor peptide of SEQ ID NO: 40 (A), with the peptide of SEQ ID NO: 50 (B) and with their combination (A+B). For comparison, the black column represents the theoretical result that was expected with the combination A+B, based on the mere addition of the effects obtained separately with A and B.

FIG. 2 shows the results of the analogous assay also described in Example 1, where the tested samples were subjected to the same treatments (A, B and A+B) and were additionally irradiated.

FIG. 3 shows the results of the in vitro repigmentation assay in reconstructed human pigmented epidermis (RHPE) described in Example 3, where the samples were treated with the combination of the MIA inhibitor peptide of SEQ ID NO: 40 (A) and the peptide of SEQ ID NO: 50 (B), at different weight ratios. In ordinates, the increase of the amount of melanin generated with each tested treatment versus the control is represented. In abscises, the different tested combinations of A+B are represented. The first column represents a weight ratio A:B of 7.5:1, the second column a weight ratio A:B of 11.2:1 and the third column, a weight ratio A:B of 22:1.

FIG. 4 shows the results of the in vitro repigmentation assay in reconstructed human pigmented epidermis (RHPE) described in Example 3. In ordinates, the increase of the amount of melanin generated with each tested treatment versus the control is represented. In abscises, the grey columns show the results of the assay for the samples treated with the MIA inhibitor peptide of SEQ ID NO: 40 (A), with the peptide of SEQ ID NO: 51 (C), and with their combination (A+C). For comparison, the black column represents the theoretical result that was expected with the combination A+C, based on the mere addition of the effects obtained separately with A and C.

FIG. 5 shows the results of the in vitro repigmentation assay in reconstructed human pigmented epidermis (RHPE) described in Example 4. In ordinates, the increase of the amount of melanin generated with each tested treatment versus the control is represented. In abscises, the grey columns show the results of the assay for the samples treated with the MIA inhibitor peptide of SEQ ID NO: 40 (A), with the peptide Pro-Pro (D) and with their combination (A+D). For comparison, the black column represents the theoretical result that was expected with the combination A+D, based on the mere addition of the effects obtained separately with A and D.

### DETAILED DESCRIPTION OF THE INVENTION

The object of the present invention is a composition comprising:
(a) a MIA inhibitor peptide of sequence SEQ ID NO: 40;
(b) at least one additional peptide selected from the group consisting of: a peptide of SEQ ID NO: 50, a peptide of SEQ ID NO: 51 and peptide Pro-Pro; and
(c) at least one pharmaceutically acceptable excipient.

Said composition is particularly suitable for the repigmentation of skin. The authors of the present invention have found that there is a significative synergistic effect in skin repigmentation when a MIA inhibitor peptide of SEQ ID NO: 40 is combined with at least another peptide selected from those of SEQ ID NOs 50, 51 and Pro-Pro. The outstanding repigmentation effect obtained with these combinations allows for an improved therapy of vitiligo, compared to the treatments available so far in art.

In the present description, as well as in the claims, the singular expressions, generally preceded by the articles “a”, “an” or “the”, are meant to include also the plural forms, unless the context clearly indicates otherwise. All percentages are expressed by weight, unless specifically stated otherwise. Numeric values preceded by the term “about” are meant to include also a certain variation around such value, namely a variation or ±5% of the stated amount. Numeric ranges defined by lower and upper endpoints are meant to include also said stated endpoints and they also include any narrower sub-range.

The amino acids are represented herein either using the one-letter code or the three-letter code, as are both well-known by the skilled in the art, according to IUPAC-IUB Commission on Biochemical Nomenclature, as described in the book Biochemical Nomenclature and Related Documents, 2nd edition, Portland Press, 1992, London [ISBN 1-85578-005-4]. Following said nomenclature, the peptides of this description are represented with the N-terminus on the left side of the formula and with the C-terminus on the right side of the formula. Some specific non-proteinogenic amino acids are represented herein as follows: D-Phenylalanine, is represented as DPhe and Norleucine is represented as Nle.

### MIA Inhibitor Peptide

A MIA inhibitor peptide is a peptide having activity as inhibitor of the human melanoma inhibitory activity (MIA) protein.

The sequence of the MIA inhibitor peptide of SEQ ID NO: 40 included in the composition of the present invention is as follows:
SEQ ID NO: 40 FHWRYPLPLPGQ

This and other MIA inhibitor peptides are disclosed in the international patent application WO-A-2012/127352, namely, the peptides having the sequences SEQ ID NO: 1 to SEQ ID NO: 49:

SEQ ID NO: 01
VPHIPPN
SEQ ID NO: 02
MPPTQVS
SEQ ID NO: 03
QMHPWPP
SEQ ID NO: 04
QPPFWQF
SEQ ID NO: 05
TPPQGLA
SEQ ID NO: 06
IPPYNTL
SEQ ID NO: 07
AVRPAPL
SEQ ID NO: 08
GAKPHPQ
SEQ ID NO: 09
QQLSPLP
SEQ ID NO: 10
GPPPSPV
SEQ ID NO: 11
LPLTPLP
SEQ ID NO: 12
QLNVNHQARADQ
SEQ ID NO: 13
TSASTRPELHYP
SEQ ID NO: 14
TFLPHQMHPWPP
SEQ ID NO: 15
VPHIPPNSMALT
SEQ ID NO: 16
RLTLLVLIMPAP
SEQ ID NO: 17
YNLPKVSSNLSP
SEQ ID NO: 18
MPPTQVSKFRLI
SEQ ID NO: 19
ANIDATPLFLRA
SEQ ID NO: 20
LLRTTETLPMFL
SEQ ID NO: 21
SALEPLV
SEQ ID NO: 22
GSPTPNA
SEQ ID NO: 23
APSHATH
SEQ ID NO: 24
TTVGHSD
SEQ ID NO: 25
THFSTFT
SEQ ID NO: 26
SLLLDTS
SEQ ID NO: 27
SVAMKAHKPLLP
SEQ ID NO: 28
NTIPGFASKSLD
SEQ ID NO: 29
VSNYKFYSTTSS
SEQ ID NO: 30
VSRHQSWHPHDL
SEQ ID NO: 31
HLNILSTLWKYR
SEQ ID NO: 32
HNASPSWGSPVM
SEQ ID NO: 33
SHPWNAQRELSV
SEQ ID NO: 34
HHWPFWRTLPLS
SEQ ID NO: 35
WHTKFLPRYLPS
SEQ ID NO: 36
NNTSFTVVPSVP
SEQ ID NO: 37
SHLSTWKWWQNR
SEQ ID NO: 38
FHWHPRLWPLPS
SEQ ID NO: 39
WHWTYGWRPPAM
SEQ ID NO: 40
FHWRYPLPLPGQ
SEQ ID NO: 41
WHWPLFIPNTTA
SEQ ID NO: 42
WHNGIWWHYGVR
SEQ ID NO: 43
HHLNYLWPWTRV
SEQ ID NO: 44
FWHRWSTFPEQP
SEQ ID NO: 45
WHMSYFWTRPPQ
SEQ ID NO: 46
FHLNWPSRADYL
SEQ ID NO: 47
WHKNTNWPWRTL
SEQ ID NO: 48
ALSPSQSHPVRS
SEQ ID NO: 49
GTQSTAIPAPTD

Within the meaning of the present invention, the MIA inhibitor peptide of sequence SEQ ID NO: 40 may be optionally acetylated at the N-terminus (CH₃—CO—) and/or amidated at the C-terminus.

C-terminal amidation includes the formation of terminal amido groups of formula —CONH₂, —CONHR or —CONR₂, wherein R is typically a short (e.g., C₁-C₄) alkyl group. The simplest and most common C-terminal amido group is —CO—NH₂.

In an embodiment, the MIA inhibitor peptide of sequence SEQ ID NO: 40 is acetylated at the N-terminus and amidated at the C-terminus.

In another embodiment, the MIA inhibitor peptide of SEQ ID NO: 40 is neither acetylated nor amidated.

The MIA inhibitor peptide can be prepared following standard methods in peptide synthesis, typically, by solid phase synthesis, or may be obtained commercially from several sources.

The composition of the invention may comprise, besides the MIA inhibitor peptide of SEQ ID NO: 40, one or more additional MIA inhibitor peptides, for example, selected from SEQ ID NOs 1-39 and 41-49.

In a preferred embodiment, the composition of the invention contains the peptide of SEQ ID NO: 40 as the only MIA inhibitor peptide of the composition.

The terms “MIA inhibitor peptide” and “MIA inhibitor” are used interchangeably herein and refer, unless the context indicates otherwise, to the MIA inhibitor peptide of SEQ ID NO: 40.

The MIA inhibitory activity can be tested using standard methods, for example, a ligand binding assay using recombinant human MIA, as discussed in Stoll et al., The extracellular human melanoma inhibitory activity (MIA) protein adopts an SH3 domain-like fold, EMBO J., 2001, 20(3), 340-349, or as disclosed in Schmidt et al., Targeting melanoma metastasis and immunosuppression with a new mode of melanoma inhibitory activity (MIA) protein inhibition, 2012, PLoS ONE 7(5): e37941.

The MIA inhibitor peptide can be added as such to the composition or may be encapsulated within different polymeric materials or conjugated to different carrier substances for improving its transdermal delivery through the stratum corneum to reach the target MIA protein in the vitiliginous melanocytes, which are located in the bottom layer of the epidermis, the stratum basale, attached to the basement membrane.

Encapsulation techniques are a well-known in the art for the delivery of active ingredients.

In an embodiment, the MIA inhibitor peptide is micro-encapsulated using liposome technology.

Liposomes are commonly used in dermal formulations for improving penetration of actives through the skin layer. As is well known in the art, liposomes are spherical vesicles with sizes generally in the range between about 60 nm and 300 nm and are most often composed of phospholipids which form at least one phospholipid bilayer, but may also include other lipids. Liposomes contain hydrophilic cores in which hydrophilic actives may be encapsulated, while hydrophobic actives are incorporated in the bilayer, so liposomes are suitable carriers for both hydrophilic and lipophilic actives (Knoth et al., Nanocarrier-Based Formulations: Production and Cosmeceutic Applications, in: Cosmetic Formulation. Principles and Practice, Benson H. A. E., Roberts M. S., Rodrigues Leite-Silva V. and Walters K. A., editors, CRC Press, 2019). Liposomes may be prepared by well-known techniques; in general, the preparation methods involve mixing the membrane-forming lipids, in an organic phase, drying, subsequent hydration of the lipids and further size reduction by different mechanical treatment such as sonication, extrusion or homogenization.

In another embodiment, the MIA inhibitor is encapsulated within a microcapsule or nanocapsule made of a biodegradable polymer, as are well-known in the art, for example, as those disclosed for the delivery of the peptides of sequences 1 to 49 (WO-A-03/064457, op.cit.) including polyesters, polyamino acids, polyalkyl cyanoacrylates, polyphosphazenes, or polyethylene oxide. Common polymers used for encapsulation are, for example, polyvinyl alcohol (PVA), poly(lactic acid) (PLA), poly(lactic-co-glycolic acid) (PLGA) or their block co-polymers with poly(ethylene oxide) or poly(ethylene glycol). Other suitable biodegradable polymers are poly(propylene fumarate-co-ethylene glycol) [P(PF-co-EG)] block copolymer, poly-anhydride poly(fumaric-co-sebacic) anhydride, alginate, dextran, chitosan, hydroxyapatite, collagen, fibrin, hyaluronic acid, carbomers, and mixtures thereof.

In another embodiment, the MIA inhibitor peptide is conjugated with nanoparticles, namely, with lipid nanoparticles, polymer nanoparticles, magnetic nanoparticles or metallic nanoparticles, which may act as carriers to facilitate the penetration of the MIA inhibitor through the stratum corneum.

In a particular embodiment, the MIA inhibitor peptide is conjugated with gold nanoparticles, which are described to improve penetration through skin (Gupta et al., J. Phys. Chem. B, 2016, 7133-7142). Those nanoparticles have a gold core coated with the MIA inhibitor peptide. The MIA inhibitor peptide is conjugated to the gold nanoparticles, typically by means of an electrostatic ionic bond between the negative charges of the gold surface of the nanoparticles and the positive charges of amino acids of the peptide carrying amino groups in the side chain (lysine, arginine, histidine, asparagine or glutamine). The gold nanoparticles have a size of less than 1000 nm, preferably comprised between 50 and 200 nm. The conjugates may be prepared by reducing Au (III) to Au (0) with a reducing agent and treating the gold nanoparticles obtained with the MIA inhibitor peptide, for example, as disclosed in the international patent application WO-A-2019/185696.

The amount of the MIA inhibitor peptide of SEQ ID: 40 in the composition of the invention, either if it is added in free form or encapsulated or conjugated with a carrier, as disclosed above, is typically comprised between 0.0001% and 1%, preferably comprised between 0.0005% and 0.1%, and more preferably comprised between 0.001% and 0.05%, wherein the percentages are expressed as weight of the MIA inhibitor peptide of SEQ ID: 40 relative to the total weight of the composition.

### Additional Peptide

The additional peptide (b) that is combined with the MIA inhibitor peptide of SEQ ID NO: 40 is selected from:
a peptide of SEQ ID NO: 50,
a peptide of SEQ ID NO: 51, and
peptide Pro-Pro;
   wherein:

SEQ ID NO: 50 is Nle-Ala-His-DPhe-Arg-Trp
SEQ ID NO: 51 is His-DPhe-Arg-Trp.

The peptide of SEQ IDE NO: 50 is disclosed, for example, in the international patent application WO-A-03/64458, for its melanotropic, anti-inflammatory and/or anti-allergic use.

Within the meaning of the present invention, the peptide of SEQ IDE NO: 50 may be optionally acetylated at the N-terminus (CH₃—CO—) and/or an amidated (—NH₂) at the C-terminus.

In an embodiment, the peptide of SEQ ID NO: 50 includes an acetyl group at the N-terminus and an amide group (—NH₂) at the C-terminus, and can be represented as Ac-Nle-Ala-His-DPhe-Arg-Trp-NH₂.

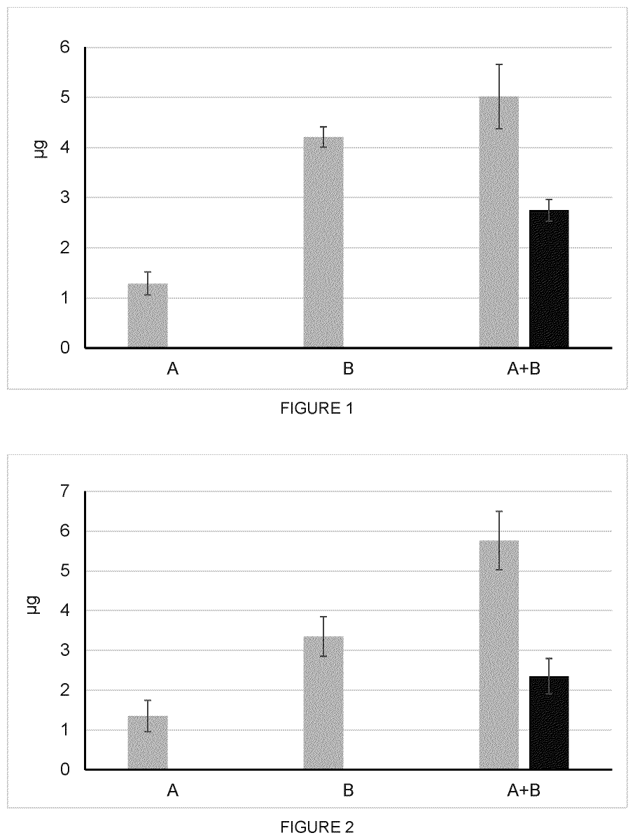

As shown in Example 1 (and in FIGS. 1 and 2) the combination of a MIA inhibitor peptide (of SEQ ID NO: 40) and the peptide of SEQ ID NO: 50 causes an outstanding increase in the production of melanin, in an in vitro assay performed with reconstructed human pigmented epidermis. The combination of both peptides provides a synergistic effect, as the increase in melanin production is clearly higher than that expected with a mere addition of the effects of the individual peptides.

The peptide of SEQ ID NO: 51 is a melanocortin agonist, as described, for example, in Holder et al, J. Med. Chem., 2002, 45 (13), 2801-2810.

Within the meaning of the present invention, the peptide of SEQ ID NO: 51 may include a N-terminus lipidation, i.e., the addition of a saturated or unsaturated C₁₂-C₁₈ fatty acid, such as myristic or palmitic, at the N-terminus, through an amide bond; and/or may be amidated (—NH₂) at the C-terminus. In an embodiment, the N-terminus of SEQ ID NO: 51 includes a palmitoyl group and the C-terminus an amide group (—NH₂).

As shown in Example 3 (and in FIG. 4) the combination of the MIA inhibitor peptide of SEQ ID NO: 40 and the peptide of SEQ ID NO: 51 causes an outstanding increase in the production of melanin, in an in vitro assay performed with reconstructed human pigmented epidermis, which is clearly higher than that expected with the mere addition of the effects of the individual peptides.

The peptide Pro-Pro is a pro-pigmenting peptide disclosed, for example, in the international patent application WO-A-2014/080376.

The peptide Pro-Pro, within the meaning of the present invention, may include a N-terminus lipidation, i.e., the addition of a saturated or unsaturated C₁₂-C₁₈ fatty acid, such as myristic or palmitic, through an amide bond; and/or the addition of an amide group (—NH₂) at the C-terminus. In an embodiment, the N-terminus of the peptide Pro-Pro includes a palmitoyl group, and it can be represented as CH₃—(CH₂)₁₄—CO-Pro-Pro (palmitoyl-prolil-proline).

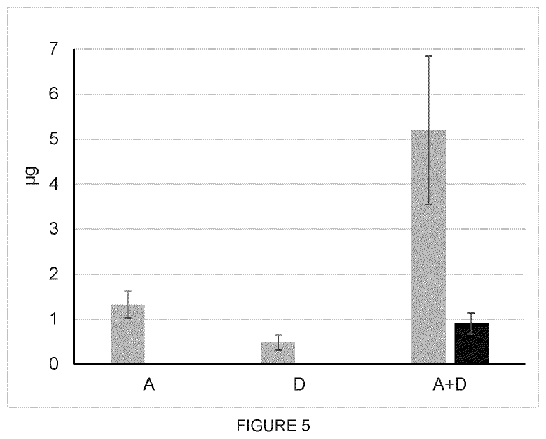

As shown in Example 4 (and in FIG. 5) the combination of the MIA inhibitor peptide of SEQ ID NO: 40 and the peptide Pro-Pro causes an outstanding increase in the production of melanin, in an in vitro assay performed with reconstructed human pigmented epidermis, which is clearly higher than that expected with the mere addition of the effects of the individual peptides.

In an embodiment, the MIA inhibitor peptide is combined with a peptide of SEQ ID NO: 50.

In an embodiment, the MIA inhibitor peptide is combined with a peptide of SEQ ID NO: 51.

In an embodiment, the MIA inhibitor peptide is combined with peptide Pro-Pro.

In an embodiment, the MIA inhibitor peptide is combined with at least two peptides selected from the group consisting of: a peptide of SEQ ID NO: 50, a peptide of SEQ ID NO: 51 and peptide Pro-Pro.

In an embodiment, the MIA inhibitor peptide is combined with a peptide of SEQ ID NO: 50, a peptide of SEQ ID NO: 51 and peptide Pro-Pro.

The weight ratio MIA inhibitor peptide of SEQ ID NO: 40 (a):additional peptide (b) in the composition of the invention is comprised between 1:1 and 100:1, preferably comprised between 2:1 and 50:1, wherein the additional peptide (b) is either the peptide of SEQ ID NO: 50 or the peptide of SEQ ID NO: 51 or the peptide Pro-Pro.

When more than one additional peptide of SEQ ID NOs 50, 51 or Pro-Pro are added to the composition, the weight ratio relates to the individual weight ratio of the MIA inhibitor peptide of SEQ ID NO: 40 to each one of the additional peptides.

When the composition of the invention comprises a peptide of SEQ ID NO: 50, the weight ratio of the MIA inhibitor peptide to the peptide of SEQ ID NO: 50 is preferably comprised between 2:1 and 50:1, more preferably comprised between 5:1 and 25:1.

When the composition of the invention comprises a peptide of SEQ ID NO: 51, the weight ratio of the MIA inhibitor peptide to the peptide of SEQ ID NO: 51 is preferably comprised between 2:1 and 60:1, more preferably comprised between 10:1 and 50:1, and still more preferably comprised between 20:1 and 40:1.

When the composition of the invention comprises a peptide Pro-Pro, the weight ratio of the MIA inhibitor peptide to the peptide Pro-Pro is preferably comprised between 1:1 and 10:1, more preferably comprised between 2:1 and 5:1.

### Optional Additional Ingredients

Optionally, the composition of the invention may contain snail secretion as additional active ingredient.

Snail secretion, also known as snail slime, is a kind of mucus or bodily secretion which is produced by snails (gastropods), by glands located in the foot of the gastropod. This mucus is made of water and a complex mixture of substances, including proteoglycans, glycosaminoglycans, glycoprotein enzymes, hyaluronic acid, copper peptides, antimicrobial peptides, and metal ions; the main components are allantoin, collagen, elastin and glycolic acid (Cillia et al., Antimicrobial properties of terrestrial snail and slug mucus, J. Complement. Integr. Med., 2018, 15(3)).

This secretion may be collected and recovered from living snails, for example as disclosed in the U.S. Pat. No. 5,538,740 or in the US patent application US-A-2018/0064635. In general, after subjecting the gastropods to certain stress conditions, the secretion is collected, and filtered.

Snail secretion is generally in the form of a liquid, the pH is generally comprised in the range 5-8, the density is typically from about 1.0 to about 1.1 mg/ml and the dry residue is generally comprised between 0.1% and 3%, preferably comprised between 0.2% and 2%, more preferably comprised between 0.25% and 1%, and still more preferably comprised between 0.3% and 0.7%. The protein content is typically in the range 0.1-3 mg/ml.

Snail secretion is commercially available from several sources, for example, from the company Cobiosa, Spain (sold as product Poly-Helixan PF) or from the company CENTISIA Laboratorio di Fitocosmesi di Bruno Dott.ssa Laura Francesca & C. s.a.s., Italy (sold as Prodotto 580038), among others.

Different species of gastropods may be used to obtain this secretion, including, among others, Achatina fulica, Cornu aspersum (or Helix aspersa or Cryptomphalus aspersa or Cantareus aspersus), Helix pomatia, Helix hortensis, Helix nemoralis, Helix cardidula, Helix tchthyomma, Helix fructicicola, Helix strigella, Helix fruticum, Helix bidens, Helix arbostorum, Helix rotundata, Helix aculeata, Helix pulchella, Helix personata, Helix holoserica, Helix aperta, Helix parnassia, Helix alonensis, Helix candidissima, Helix pisana, or Helix gualteviana. In a particular embodiment, the snail secretion is obtained from Cornu aspersum, also known as Helix aspersa, Cryptomphalus aspersa or Cantareus aspersus.

The medicinal and cosmetic use of snail secretion has been disclosed in the state of the art, for example, as antimicrobial agent, for wound repair, for skin protection, or as anti-aging agent, among other uses.

The combined use of a MIA inhibitor peptide and snail secretion is disclosed in the international patent application WO-A-2022/008251.

The amount of snail secretion in the composition of the invention, if present, is typically comprised between 0.5% and 15%, preferably comprised between 1% and 10%, more preferably comprised between 2% and 8% and still more preferably is about 5%, wherein the percentages are expressed as weight of the snail secretion relative to the total weight of the composition.

### Compositions

The composition of the invention comprises at least one pharmaceutically acceptable excipient.

The term “pharmaceutically acceptable excipient” means a component of a pharmaceutical or cosmetic composition that is not an active ingredient, which is useful for preparing the composition and is generally safe and non-toxic for human pharmaceutical or cosmetic use. In particular, the composition of the invention is typically intended to be used externally, topically applied over the affected skin area, so the pharmaceutically acceptable excipient is specifically “dermatologically acceptable”, i.e., it is suitable and non-toxic for use in contact with human skin tissue.

In an embodiment, the composition according to present invention is for topical administration, i.e., it is to be spread over the skin in the area to be treated, namely, in the area showing depigmentation, typically vitiliginous depigmentation, or prone to show depigmentation. Topical, dermal, or cutaneous administration are used herein interchangeably.

Any type of formulation suitable for topical administration may be used. Typically, the composition of the invention is in the form of cream, gel, cremigel, lotion, paste, foam, solution, suspension, emulsion, milk, or stick preparation, for example, which are well-known in the art. A cremigel, for example, is a type of formulation halfway between a cream and a gel: unlike a cream, a cremigel is a gelled aqueous solution or a gelled oil, but it looks opaque as a cream, usually white in colour.

Suitable carriers may be, for example, anhydrous, as mixtures of fats, waxes, animal and plant oils and solid and liquid hydrocarbons. Or the carrier may be water or an aqueous solution of hydrophilic substances. Suitably, the carrier may be in the form of an emulsion. Emulsions may be, typically, oil-in-water emulsions, water-in-oil emulsions, water-in-oil-in-water, oil-in-water-in-oil or water-in-silicone emulsions. An emulsion may generally be described as having a continuous aqueous phase (oil-in-water and water-in-oil-in-water) or a continuous oil phase (water-in-oil and oil-in-water-in-oil). The oil phase may comprise silicone oils, non-silicone oils such as paraffin hydrocarbons, fatty alcohols (e.g. stearyl alcohol, cetyl alcohol or cetostearyl alcohol), fatty acids (e.g. stearic acid, oleic acid), fatty acid esters (e.g. isopropyl myristate, isopropyl palmitate), waxes or plant oils (e.g. castor oil, canola oil, cottonseed oil, jojoba oil or arachis oil), or mixtures thereof. The aqueous phase may comprise water or a water solution of hydrophilic substances, such as alcohols (e.g. ethanol, isopropyl alcohol), polyols (e.g. glycerol, sorbitol), alpha hydroxy acids, amino acids, protein hydrolysates, simple sugars, or polysaccharides.

Emulsifiers, which are common components of emulsions, are surface-active agents (surfactants) and include non-ionic surfactants, anionic surfactants, cationic surfactants and amphoteric surfactants. Non-ionic surfactants include, among others, ethoxylated fatty alcohols, ethoxylated fatty acid esters, alkyl glucosides or alkyl oligoglucosides, ethoxylated sorbitan fatty acid esters, monoglycerol/polyglycerol fatty acid esters, ethoxylated glycerin monesters, ethoxylated polyglyceryl esters, alkyl dimethylamine oxides, or poloxamers, among others. Anionic surfactants include alkaline soaps, alkyl sulphates, alkyl ether sulphates, alkyl sulphosuccinates, alkyl phosphates, acyl sarcosinates or acyl isethionates, among others. Cationic surfactants include quaternary ammonium salts, or pyridine salts, among others. Amphoteric surfactants include imidazoline derivatives, betaines, amidobetaines and sulphobetaines.

Other common ingredients in the formulation are, for example, emollients, humectants, preservatives, viscosity controlling agents, antioxidants, pH regulators, UV filters, chelating agents, perfumes and colorants. Common emollients are, for example, paraffin hydrocarbons, silicones, fatty alcohols, fatty acids, esters of fatty acids with alcohols, triglycerides, ceramides, phospholipids and waxes. Humectants include polyhydroxy alcohols, proteins and hydroxyl acids. Common preservatives include sorbic acid and its salts, benzoic acid and its salts, parabens, imidazolidinyl urea, diazolidinyl urea, DMDM hydantoin, sodium hydroxymethylglycinate, methylchloroisothiazolinone/methylisothiazolinone, benzyl alcohol and 2-phenoxyethanol, among others. Other additives may also be added for controlling the viscosity of the formulation, for example, xanthan gum, gellan gum, acacia, carrageenans, chitosan, collagen, tragacanth, pectin, starch derivatives, carbomers, cellulose derivatives (hydroxyethylcellulose, hydroxypropylcellulose, hydroxypropyl methylcellulose, methylcellulose, ethylcellulose, or carboxymethylcellulose, for example), polyamides, glutamides, colloidal silica or waxes (e.g. beeswax or vegetable waxes), among others.

Some ingredients of the composition may act as penetration enhancers, to facilitate the penetration of the active ingredients throughout the stratum corneum. Common penetration enhancers are, for example, short chain alcohols such as ethanol or isopropyl alcohol; long chain alcohols such as decanol, hexanol, lauryl alcohol, myristyl alcohol, octanol, octyldodecanol or oleyl alcohol; cyclic amides such as azone; esters such as ethyl acetate octyl salicylate, padimate O, ethyl oleate, glyceryl monoleate, glyceryl monocaprate, glyceryl tricaprylate, isopropyl myristate, isopropyl palmitate, propylene glycol monolaurate, or propylene glycol monocaprylate; ether alcohols such as Transcutol®; fatty acids such as lauric acid, linoleic acid, linolenic acid, myristic acid, oleic acid, palmitic acid, stearic acid or isostearic acid; glycols such as dipropylene glycol, propylene glycol, 1,2-butylene glycol or 1,3-butylene glycol; pyrrolidones such as N-methyl-2-pyrrolidone or 2-pyrrolidone; sulphoxides such as decylmethyl sulphoxide or dimethyl sulphoxide; anionic surfactants; cationic surfactants; non-ionic surfactants; terpenes such as eugenol D-limonene, menthol, menthone, farnesol or neridol (as disclosed, for example, in Lane M. E., Skin penetration enhancers, Int. J. Pharm., 2013, 447, 12-21; or in Haque et al., Chemical enhancer: a simplistic way to modulate barrier function of the stratum corneum, Adv. Pharm. Bull., 2018, 8(2), 169-179).

The composition of the invention has typically a slightly acidic pH, close to the physiological pH of the skin. Common acidity regulators are organic acids, including hydroxy acids and fatty acids. Some of the most common pH regulators in cosmetic emulsions are hydroxy acids such as lactic or citric acid.

One excipient can perform more than one function. The above cited ingredients, as well as many others suitable dermatological formulation excipients, are well-known to the skilled formulator of topical compositions. Such ingredients are commercially available from several companies, such as Comercial Quimica Massó, SA, Evonik, DuPont or Dow Corning, among others.

The preparation of the topical composition is made according to procedures well-known to the skilled in the formulation of dermatological compositions, generally involving simple steps of mixing, and optionally heating the component ingredients.

A cream, for example, can be typically prepared by warming the oily phase and the aqueous phase separately to a temperature of about 60 to 80° C., and then mixing under stirring to prepare the emulsion.

The main ingredients of the compositions and the procedures for preparing them are described, for example, in the book: Cosmetic Formulation. Principles and Practice, Benson H. A. E., Roberts M. S., Rodrigues Leite-Silva V. and Walters K. A., editors, CRC Press, 2019, or in other similar reference books. Also, regulated cosmetic substances and ingredients are disclosed in the European Commission database “Cosing” (https://ec.europa.eu/growth/tools-databases/cosing).

Typically, for example, a cream, gel, or lotion base formulation is first prepared, using standard ingredients and procedures well-known in the art, and then the MIA inhibitor peptide, the additional peptide, and snail secretion (if present) are added and thoroughly mixed, and the pH of the composition is finally adjusted.

### Use of the Composition

As shown in the in vitro repigmentation assays of the examples, the combination of the MIA inhibitor peptide and the additional peptide, as defined above, is outstandingly effective for skin repigmentation.

Another aspect of the invention is, therefore, the composition of the invention for use in the prevention and/or treatment of vitiligo.

Another aspect of the invention is a method for the prevention and/or treatment of vitiligo in a patient in need thereof comprising the steps of applying a therapeutically effective amount of the composition of the invention.

The composition of the invention can also be used for the cosmetic, non-therapeutic repigmentation of small areas of depigmented, whitened skin, which may not be specifically defined as vitiliginous.

Therefore, another aspect of the invention is the cosmetic, non-therapeutic use of the composition of the invention for skin repigmentation.

Another aspect of the invention is a method for skin repigmentation in a subject in need thereof comprising the steps of applying an effective amount of the composition of the invention.

The cosmetic use for skin repigmentation is understood as the procedure aimed to partially or totally restore the normal pigmentation of the depigmented or hypopigmented skin patches in a human subject.

Analogously, the treatment of vitiligo is understood as the procedure aimed to improve vitiligo symptoms, namely, to partially or totally restore the normal pigmentation of the depigmented or hypopigmented skin patches in a human subject.

In an embodiment, the prevention and/or treatment of vitiligo relates to the prevention and/or treatment of non-segmental vitiligo.

The prevention of vitiligo is aimed to prevent the onset of vitiligo symptoms and is addressed to subjects which are known to be prone to suffer from vitiligo, either for having been early diagnosed, before the appearance of symptoms, for example, by detecting the presence of MIA protein in the melanocytes, or for having some known genetic predisposition to suffer from this disease. The prevention of vitiligo also includes the treatment of patients who had previously suffered from vitiligo and have recovered from the disease, i.e., who have achieved re-pigmentation of the vitiliginous depigmented skin, to prevent them from suffering again from vitiligo symptoms.

Typically, the composition of the invention is administered topically, i.e., it is spread over the vitiliginous or depigmented skin patches.

In an embodiment, the composition of the invention may be delivered transdermally, for example, via iontophoresis, ultrasound or using microneedles (Escobar-Chávez et al. Microneedles: a valuable physical enhancer to increase transdermal drug delivery, J. Clin. Pharmacol., 2011, 51(7), 964-977).

The amount of the composition to be used, considered to be “effective” for the treatment or prevention of vitiligo or for cosmetic, non-therapeutic skin repigmentation can widely vary depending on many factors, for example, the severity and progression stage of the depigmentation condition, or the specific formulation used. The amount can be easily adjusted in each case by the skilled practitioner. In general, the amount of the topical composition applied to the affected skin may range from about 0.1 mg of composition for each cm² of skin surface area (mg/cm²) to about 100 mg/cm², preferably from about 5 mg/cm² to about 10 mg/cm².

The compositions of the present invention may be applied topically once or more times daily, for example twice, three times or four times daily. The composition is typically applied by spreading it over the skin, generally, only over the affected skin area. The duration of the treatment may be adjusted according to the repigmentation evolution. Typically, the treatment may be maintained for several weeks, for example 1, 2, 3 or 4 weeks, or several months, for example, 1 to 12 months, or even more, depending on the severity of depigmentation symptoms and the evolution of the condition. The duration of the treatment can be easily adjusted by the skilled practitioner.

Advantageously, the treatment with the composition of the invention may be combined with phototherapy, for example, by simple sunlight exposure or by the use of sunbeds, to stimulate melanocyte formation and migration and thus boosting the repigmentation.

Another aspect of the invention is, therefore, a method for the prevention and/or treatment of vitiligo in a patient in need thereof comprising the steps of applying a therapeutically effective amount of the composition of the invention and exposing the treated skin area to phototherapy.

The term “phototherapy” or “light therapy” as used herein includes exposure to sunlight or to radiation of specific wavelengths, for example, UV radiation, including UVA and UVB radiation. Therefore, “phototherapy” can be performed by simple exposure to sunlight, or using sunbeds, or using a radiation emitting device, in particular, a UV emitting device.

The duration of the phototherapy is not decisive, and may range, for example, from about 2 minutes to about 2 hours daily, or on alternate days, or twice or three times weekly, among other suitable options.

Vitiligo, or skin depigmentation in general, can affect any area of skin, so the composition may be applied to any area of affected (depigmented) skin. The areas of skin most commonly affected and, therefore, more in need to be treated, are hands and face, around body openings (the eyes, nostrils, mouth, umbilicus and genital regions), and within body folds such as the underarms and groin.

In one embodiment, the use of the composition of the invention for treating skin depigmentation in general, or vitiligo in particular, may be combined with the use of antioxidants, in particular, when the therapy is combined with phototherapy, in order to reduce the possible negative effects of the radiation exposure.

The antioxidants are typically for oral use. Suitable antioxidants are, among others, vitamin E, vitamin C, carotenoids (such as lycopene), beta-carotene, alpha-carotene, green tea extract, zinc, selenium, Polypodium leucotomos extract, among others, or combinations thereof.

They can be formulated as a dietary oral supplement, typically as tablets, powders or capsules, for example, using standard excipients and preparation process, known in the art.

Another aspect of the invention is also a method for the prevention and/or treatment of vitiligo in a patient in need thereof comprising the steps of applying a therapeutically effective amount of the composition of the invention, exposing the treated skin area to phototherapy and administering to the patient an antioxidant.

The present invention may be defined according to the following embodiments:

1. A composition comprising:
(a) a MIA inhibitor peptide of sequence SEQ ID NO: 40;
(b) at least one additional peptide selected from the group consisting of: a peptide of SEQ ID NO: 50, a peptide of SEQ ID NO: 51 and peptide Pro-Pro; and
(c) at least one pharmaceutically acceptable excipient.
   2.—The composition according to embodiment 1, characterized in that the MIA inhibitor peptide of SEQ ID NO: 40 is acetylated at the N-terminus and/or amidated at the C-terminus; and more preferably it is acetylated at the N-terminus and amidated at the C-terminus.
   3.—The composition according to embodiment 2, characterized in that C-terminal amide group is selected from —CONH₂, —CONHR and —CONR₂, wherein R is C₁-C₄ alkyl group, and preferably is —CO—NH₂.
   4.—The composition according to embodiment 1, characterized in that the MIA inhibitor peptide of SEQ ID NO: 40 is neither acetylated nor amidated.
   5.—The composition according to any one of embodiments 1 to 4, characterized in that the MIA inhibitor peptide is encapsulated or is conjugated with a carrier nanoparticle.
   6.—The composition according to any one of embodiments 1 to 5, characterized in that the amount of the MIA inhibitor peptide in the composition is comprised between 0.0001% and 1%, preferably comprised between 0.0005% and 0.1%, and more preferably comprised between 0.001% and 0.05%, wherein the percentages are expressed as weight of the MIA inhibitor peptide relative to the total weight of the composition.
   7.—The composition according to any one of embodiments 1 to 6, characterized in that it comprises a peptide of SEQ ID NO: 50.
   8.—The composition according to embodiment 7, characterized in that the peptide of SEQ ID NO: 50 is acetylated at the N-terminus (CH₃—CO—) and/or an amidated (—NH₂) at the C-terminus, preferably is acetylated at the N-terminus and amidated at the C-terminus (Ac-Nle-Ala-His-DPhe-Arg-Trp-NH₂).
   9.—The composition according to any one of embodiments 1 to 8, characterized in that it comprises a peptide of SEQ ID NO: 51.
   10.—The composition according to embodiment 9, characterized in that the N-terminus of the peptide of SEQ ID NO: 51 includes a palmitoyl group and the C-terminus an amide group (—NH₂).
   11. The composition according to any one of embodiments 1 to 10, characterized in that it comprises peptide Pro-Pro.
   12.—The composition according to embodiment 11, characterized in that the N-terminus of the peptide Pro-Pro includes a palmitoyl group (palmitoyl-prolil-proline).
   13.—The composition according to any one of embodiments 1 to 12, characterized in that the weight ratio of the MIA inhibitor peptide (a) to the additional peptide (b) is comprised between 1:1 and 100:1, preferably comprised between 2:1 and 50:1.
   14.—The composition according to embodiments 7 or 8, characterized in that the weight ratio of the MIA inhibitor peptide (a) to the peptide of SEQ ID NO: 50 is comprised between 2:1 and 50:1, preferably comprised between 5:1 and 25:1.
   15.—The composition according to embodiments 9 or 10, characterized in that the weight ratio of the MIA inhibitor peptide (a) to the peptide of SEQ ID NO: 51 is comprised between 2:1 and 60:1, preferably comprised between 10:1 and 50:1, and more preferably comprised between 20:1 and 40:1.
   16.—The composition according to embodiments 11 or 12, characterized in that the weight ratio of the MIA inhibitor peptide (a) to the peptide Pro-Pro is comprised between 1:1 and 10:1, preferably comprised between 2:1 and 5:1.
   17.—The composition according to any one of embodiments 1 to 16, characterized in that the composition comprises snail secretion.
   18.—The composition according to embodiment 17, characterized in that the amount of snail secretion in the composition is comprised between 0.5% and 15%, preferably comprised between 1% and 10%, more preferably comprised between 2% and 8% and still more preferably is about 5%, wherein the percentages are expressed as weight of the snail secretion relative to the total weight of the composition.
   19.—The composition according to any one of embodiments 1 to 18, characterized in that it is for topical administration.
   20.—The composition according to embodiment 19, characterized in that it is selected from cream, gel, cremigel, lotion, paste, foam, solution, suspension, emulsion, milk, and stick, preferably is selected from cream, gel and cremigel, more preferably is selected from cream and gel.
   21.—The composition according to any one of embodiments 1 to 20 for use in the prevention and/or treatment of vitiligo.
   22.—The composition for use according to embodiment 21, characterized in that vitiligo is non-segmental vitiligo.
   23.—Cosmetic, non-therapeutic use of the composition according to any one of embodiments 1 to 20 for skin repigmentation.
   24.—A method for the prevention and/or treatment of vitiligo in a patient in need thereof comprising the steps of applying a therapeutically effective amount of the composition according to any one of embodiments 1 to 20.
   25.—A method for the prevention and/or treatment of vitiligo in a patient in need thereof comprising the steps of applying a therapeutically effective amount of the composition according to any one of embodiments 1 to 20 and exposing the treated skin area to phototherapy.
   26.—The method according to embodiment 25, wherein phototherapy includes exposure to sunlight or to radiation of specific wavelengths, for example, UV radiation, including UVA and UVB radiation.
   27.—The method according to embodiments 25 or 26, wherein the method includes an additional step of administering to the patient an antioxidant.
   28.—The method according to any one of embodiments 24 to 25, wherein vitiligo is non-segmental vitiligo.

### EXAMPLES

### Example 1: In Vitro Repigmentation Assay in Reconstructed Human Pigmented Epidermis (RHPE) Treated with the Combination of a MIA Inhibitor Peptide and a Peptide of SEQ ID 50

For assessing the repigmentation effect, a reconstructed epidermis model was used, namely, based on adding melanocytes and keratinocytes on a polycarbonate filter (SkinEthic® RHPE, available from the company Episkin).

Sample tissues were treated with the test products for 6 days (from day1 to day6). After each dosing, the samples were incubated at 37° C., and melanin extraction was performed after 6 days of treatment (at day7). Untreated samples were also included in the assay as controls. An evaluation of cell viability was also performed.

After treatment, melanin extraction was performed by plunging each sample filter into 800 μl of Solvable® (Perkin Elmer) and heating at 63° C. overnight. The optical density of the supernatants was measured on 300 μl extract at 405 nm using synthetic melanin as reference. 6 samples were used for each treatment and the mean value was calculated.

The tested treatments were the following:

| | | Concentration in |
|---|---|---|
| | Treatment | culture medium |
| 0 | Control (no treatment) | |
| A | MIA inhibitor of SEQ ID NO: 40 | 3.0 ppm |
| B | Peptide of SEQ ID NO: 50 | 0.4 ppm |
| A + B | MIA Inhibitor of SEQ ID NO: 40 plus | 1.5 ppm + 0.2 ppm |
| | Peptide of SEQ ID NO: 50 | (7.5:1) |

The Peptide of SEQ ID NO: 50 used in this assay was acetylated at the N-terminus.

Another analogous assay, with the same treatments, was performed but simultaneously irradiating the samples. In this second assay, the samples were irradiated with 150 J/m² at 300 W/m² three times on consecutive days (on day2, day3, and day4 of the assay).

For each of the treatments the increase in the amount of melanin generated versus the control (0) was calculated. For analysing a possible synergistic effect, the experimental increase in melanin in the combined treatment (A+B) was compared to the theoretical increase to be expected if a simple additive effect were obtained.

The results are represented below (median values of the 6 samples used, SE means Standard Error):

| | Melanin | Melanin increase | | Theoretical | |
|---|---|---|---|---|---|
| | (μg) | vs. control (μg) | SE | (additive effect) | SE |
| 0 | 33.10 | — | | | |
| A | 34.39 | 1.29 | 0.23 | — | |
| B | 37.31 | 4.21 | 0.20 | — | |
| A + B | 38.12 | 5.02 | 0.64 | 2.75 | 0.22 |

The results of the above table are represented in FIG. 1, wherein the grey bars represent the experimental results and the black bar the theoretical result for the combination.

The results of the assay with irradiation of the samples are shown in the following table (median values of the 6 samples used):

| | Melanin | Melanin increase | | Theoretical | |
|---|---|---|---|---|---|
| | (μg) | vs. control (μg) | SE | (additive effect) | SE |
| 0 | 34.72 | — | | | |
| A | 36.07 | 1.35 | 0.40 | — | |
| B | 38.07 | 3.35 | 0.50 | — | |
| A + B | 40.48 | 5.76 | 0.74 | 2.35 | 0.45 |

The results of the above table are represented in FIG. 2, wherein the grey bars represent the experimental results and the black bar the theoretical result for the combination.

In both assays, either with irradiation or without irradiation, a synergistic effect in the increase of production of melanin is observed due to the combination of both peptides, because the increase obtained with the combination is clearly higher than the expected increase due to the addition of the individual effects of both peptides.

### Example 2: In Vitro Repigmentation Assay in Reconstructed Human Pigmented Epidermis (RHPE) Treated with the Combination of a MIA Inhibitor Peptide and a Peptide of SEQ ID: 50, at Different Ratios

Another in vitro repigmentation assay was performed using the same reconstructed epidermis model and the same procedure as disclosed in Example 1. In this case, the assay was performed without irradiation.

The treatments assayed were the combination of the same peptides as in Example 1, but using different weight ratios between them, as summarized in the following table:

| | | Concentration |
|---|---|---|
| | | in culture |
| | Treatment | medium |
| 0 | Control (no treatment) | |
| A + B_1 | MIA Inhibitor of SEQ ID NO: 40 plus | 1.5 ppm + 0.2 ppm |
| | Peptide of SEQ ID NO: 50 | (7.5:1) |
| A + B_2 | MIA Inhibitor of SEQ ID NO: 40 plus | 1.5 ppm + 0.13 ppm |
| | Peptide of SEQ ID NO: 50 | (11.2:1) |
| A + B_3 | MIA Inhibitor of SEQ ID NO: 40 plus | 1.5 ppm + 0.07 ppm |
| | Peptide of SEQ ID NO: 50 | (22:1) |

The results are represented below (median values of the 6 samples used):

| | | Melanin increase | |
|---|---|---|---|
| | Melanin (μg) | vs. control (μg) | SE |
| 0 | 24.29 | — | |
| A + B_1 | 27.34 | 3.05 | 0.30 |
| A + B_2 | 27.46 | 3.17 | 0.15 |
| A + B_3 | 28.18 | 3.89 | 0.28 |

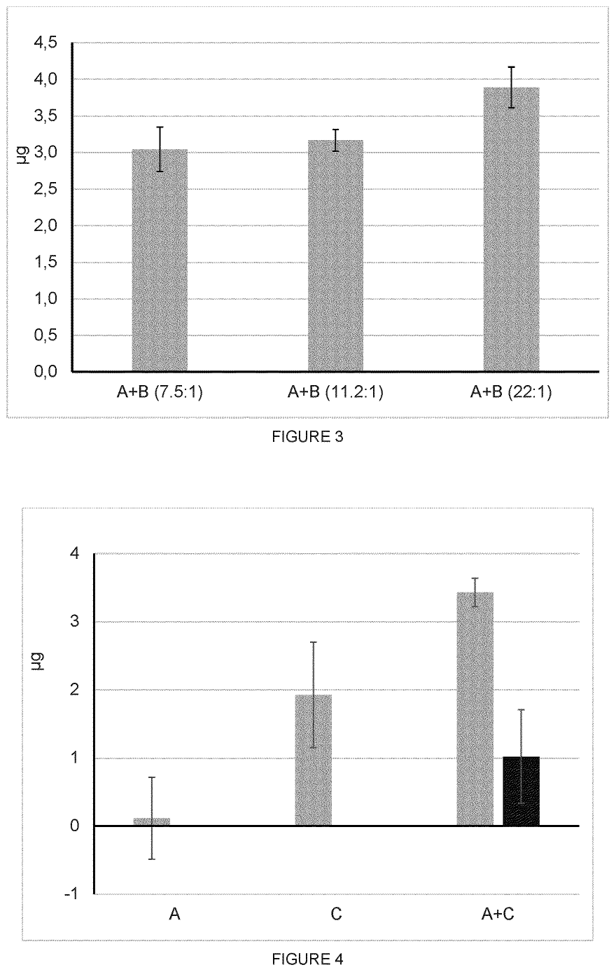

The results of the above table are represented in FIG. 3.

### Example 3: In Vitro Repigmentation Assay in Reconstructed Human Pigmented Epidermis (RHPE) Treated with the Combination of a MIA Inhibitor Peptide and a Peptide of SEQ ID: 51

In this example, the same reconstructed epidermis model was used, namely, based on adding melanocytes and keratinocytes on a polycarbonate filter (SkinEthic® RHPE, available from the company Episkin), using the same protocol disclosed in Example 1.

The tested treatments were the following:

| | | Concentration in |
|---|---|---|
| | Treatment | culture medium |
| 0 | Control (no treatment) | |
| A | MIA inhibitor of SEQ ID NO: 40 | 3.0 ppm |
| C | Peptide of SEQ ID NO: 51 | 0.1 ppm |
| A + C | MIA Inhibitor of SEQ ID NO: 40 plus | 1.5 ppm + 0.05 ppm |
| | Peptide of SEQ ID NO: 51 | (30:1) |

For each of the treatments the increase in the amount of melanin generated versus the control (0) was calculated. For analysing a possible synergistic effect, the experimental increase in melanin in the combined treatment (A+C) was compared to the theoretical increase to be expected if a simple additive effect were obtained.

The results are represented below (median values of the 6 samples used):

| | Melanin | Melanin increase | | Theoretical | |
|---|---|---|---|---|---|
| | (μg) | vs. control (μg) | SE | (additive effect) | SE |
| 0 | 36.52 | — | | — | |
| A | 36.63 | 0.11 | 0.60 | — | |
| C | 38.45 | 1.93 | 0.78 | — | |
| A + C | 39.95 | 3.43 | 0.21 | 1.02 | 0.69 |

The results of the above table are represented in FIG. 4, wherein the grey bars represent the experimental results and the black bar represents the theoretical value expected for a simple additive effect.

It is again observed a synergistic effect due to the combination of both peptides in the increase of melanin production, because the increase of the combination is clearly higher than the expected increase due to the addition of the effects of both peptides.

An analogous assay with irradiation was also performed, with similar results.

### Example 4: In Vitro Repigmentation Assay in Reconstructed Human Pigmented Epidermis (RHPE) Treated with the Combination of a MIA Inhibitor Peptide and Peptide Pro-Pro

In this example, the same reconstructed epidermis model was used, namely, based on adding melanocytes and keratinocytes on a polycarbonate filter (SkinEthic® RHPE, available from the company Episkin), using the same protocol disclosed in Example 1.

The tested treatments were the following:

| | | Concentration in |
|---|---|---|
| | Treatment | culture medium |
| 0 | Control (no treatment) | |
| A | MIA inhibitor of SEQ ID NO: 40 | 3.0 ppm |
| D | Peptide Pro-Pro | 1.2 ppm |
| A + D | MIA Inhibitor of SEQ ID NO: 40 plus | 1.5 ppm + 0.6 ppm |
| | Peptide Pro-Pro | (2.5:1) |

In particular, the peptide Pro-Pro used in this assay carried a palmitoyl group at the N-terminus, i.e., it was palmitoyl-prolil-proline.

For each of the treatments the increase in the amount of melanin generated versus the control (0) was calculated. For analysing a possible synergistic effect, the experimental increase in melanin in the combined treatment (A+D) was compared to the theoretical increase to be expected if a simple additive effect were obtained.

The results are represented below (median values of the 6 samples used):

| | Melanin | Melanin increase | | Theoretical | |
|---|---|---|---|---|---|
| | (μg) | vs. control (μg) | SE | (additive effect) | SE |
| 0 | 32.01 | — | | — | |
| A | 33.34 | 1.33 | 0.30 | — | |
| D | 32.49 | 0.48 | 0.17 | — | |
| A + D | 37.21 | 5.2 | 1.65 | 0.90 | 0.24 |

The results of the above table are represented in FIG. 5, wherein the grey bars represent the experimental results and the black bar represents the theoretical value expected for a simple additive effect.

It is again observed a synergistic effect in the increase of melanin production due to the combination of both peptides, because the combination provides a clearly higher increase of melanin production than the expected increase due to the addition of the effects of both peptides.

### Example 5 Preparation of Compositions According to the Invention

Four compositions (A, B, C and D) according to the present invention were prepared using the components listed in the following table:

| | | Weight % | | | |
|---|---|---|---|---|---|
| Ingredients | | A | B | C | D |
| A1 | Emulium ® Mellifera MB | 5.0 | 5.0 | 5.0 | 5.0 |
| | (polyglyceryl-6 distearate, jojoba esters, | | | | |
| | polyglyceryl-3 beeswax and cetyl alcohol) | | | | |
| A2 | Isohexadecane | 2.0 | 2.0 | 2.0 | 2.0 |
| A3 | Isopropyl isostearate | 1.0 | 1.0 | 1.0 | 1.0 |
| A4 | Isononyl Isononanoate | 2.0 | 2.0 | 2.0 | 2.0 |
| A5 | Tocopheryl acetate | 0.2 | 0.2 | 0.2 | 0.2 |
| B1 | Deionized water | 70.9 | 65.9 | 70.4 | 68.5 |
| B2 | Potassium cetyl phosphate | 0.3 | 0.3 | 0.3 | 0.3 |
| B3 | Anhydrous betaine extracted from sugar | 2.0 | 2.0 | 2.0 | 2.0 |
| | beet | | | | |
| B4 | Preservatives (methylpropanediol, | 2.0 | 2.0 | 2.0 | 2.0 |
| | caprylyl glycol and phenylpropanol) | | | | |
| B5 | Phenoxyethanol | 0.5 | 0.5 | 0.5 | 0.5 |
| B6 | Disodium EDTA | 0.1 | 0.1 | 0.1 | 0.1 |
| B7 | Glycerin | 2.0 | 2.0 | 2.0 | 2.0 |
| B8 | Xanthan gum | 0.2 | 0.2 | 0.2 | 0.2 |
| C1 | SEPIPLUS ™ 400 | 0.5 | 0.5 | 0.5 | 0.5 |
| | (Polyacrylate-13, Polyisobutene and | | | | |
| | Polysorbate 20) | | | | |
| C2 | Cyclopentasiloxane | 2.0 | 2.0 | 2.0 | 2.0 |
| C3 | Dimethicone | 1.0 | 1.0 | 1.0 | 1.0 |
| D1 | Snail secretion | — | 5.0 | 5.0 | 5.0 |
| D2 | Anti-MIA peptide (0.1 wt %) | 5.0 | 5.0 | 1.5 | 5.0 |
| D3a | Peptide of SEQ ID NO: 50 (0.02 wt %) | 3.0 | 3.0 | — | — |
| D3b | Peptide of SEQ ID NO: 51 (0.002 wt %) | — | | 2.0 | — |
| D3c | Palmitoyl-prolil-proline (0.6 wt %) | — | — | — | 0.4 |
| D4 | Perfume | 0.3 | 0.3 | 0.3 | 0.3 |
| | Total | 100.0 | 100.0 | 100.0 | 100.0 |

The peptide components of the composition (D2, D3a, D3b and D3c) were added as aqueous or aqueous/glycerin solutions comprising about 0.1 wt %, about 0.02 wt %, about 0.002 wt % and about 0.6 wt %, respectively, of said peptides.

For preparing the composition, first components A1-A6 were heated to about 70° C. to 75° C. and thoroughly mixed to obtain a first liquid phase (“phase A”). Components B1-B8 were separately mixed to form a solution, which was heated to about 70° C. to 75° C. Phase A was added to this solution and was emulsified in a high-shear Ultra-Turrax mixer at 8000 rpm for about 3 minutes and the emulsion was then stirred in a blade mixer at 300 rpm for about 15 minutes. The components C1-C3 were added at 60° C., the mixture was allowed to cool down to room temperature and components D1, D2, D3a/D3b/D3c and D4 were finally added. The final pH of the composition was checked to be in the range 5.5-6.5 (or otherwise adjusted with 10% citric acid or 10% NaOH).

1. A composition comprising:
(a) a MIA inhibitor peptide of SEQ ID NO: 40;
(b) at least one additional peptide selected from the group consisting of: a peptide of SEQ ID NO: 50, a peptide of SEQ ID NO: 51 and peptide Pro-Pro; and
(c) at least one pharmaceutically acceptable excipient.

2. The composition according to claim 1, characterized in that the amount of the MIA inhibitor peptide in the composition is comprised between 0.0001% and 1%, preferably comprised between 0.0005% and 0.1%, and more preferably comprised between 0.001% and 0.05%, wherein the percentages are expressed as weight of the MIA inhibitor peptide relative to the total weight of the composition.

3. The composition according to claim 1 or 2, characterized in that it comprises a peptide of SEQ ID NO: 50.

4. The composition according to any one of claims 1 to 3, characterized in that it comprises a peptide of SEQ ID NO: 51.

5. The composition according to any one of claims 1 to 4, characterized in that it comprises peptide Pro-Pro.

6. The composition according to any one of claims 1 to 5, characterized in that the weight ratio of the MIA inhibitor peptide (a) to the additional peptide (b) is comprised between 1:1 and 100:1.

7. The composition according to claim 6, characterized in that the weight ratio of the MIA inhibitor peptide (a) to the additional peptide (b) is comprised between 2:1 and 50:1.

8. The composition according to any one of claims 1 to 7, characterized in that the composition comprises snail secretion.

9. The composition according to claim 8, characterized in that the amount of snail secretion in the composition is comprised between 0.5% and 15%, preferably comprised between 1% and 10%, more preferably comprised between 2% and 8% and still more preferably is about 5%, wherein the percentages are expressed as weight of the snail secretion relative to the total weight of the composition.

10. The composition according to any one of claims 1 to 9, characterized in that it is for topical administration.

11. The composition according to claim 10, characterized in that it is selected from cream, gel, cremigel, lotion, paste, foam, solution, suspension, emulsion, milk, and stick.

12. The composition according to claim 11, characterized in that it is selected from cream, gel and cremigel, and preferably is selected from cream and gel.

13. The composition according to any one of claims 1 to 12 for use in the prevention and/or treatment of vitiligo.

14. The composition for use according to claim 13, characterized in that vitiligo is non-segmental vitiligo.

15. Cosmetic, non-therapeutic use of the composition according to any one of claims 1 to 12 for skin repigmentation.


SEQUENCE LISTING (51 sequences)

SEQ ID NO: 1 - protein, 7 aa, synthetic construct
  REGION 1..7: note = MIA inhibitor
VPHIPPN

SEQ ID NO: 2 - protein, 7 aa, synthetic construct
  REGION 1..7: note = MIA inhibitor
MPPTQVS

SEQ ID NO: 3 - protein, 7 aa, synthetic construct
  REGION 1..7: note = MIA inhibitor
QMHPWPP

SEQ ID NO: 4 - protein, 7 aa, synthetic construct
  REGION 1..7: note = MIA inhibitor
QPPFWQF

SEQ ID NO: 5 - protein, 7 aa, synthetic construct
  REGION 1..7: note = MIA inhibitor
TPPQGLA

SEQ ID NO: 6 - protein, 7 aa, synthetic construct
  REGION 1..7: note = MIA inhibitor
IPPYNTL

SEQ ID NO: 7 - protein, 7 aa, synthetic construct
  REGION 1..7: note = MIA inhibitor
AVRPAPL

SEQ ID NO: 8 - protein, 7 aa, synthetic construct
  REGION 1..7: note = MIA inhibitor
GAKPHPQ

SEQ ID NO: 9 - protein, 7 aa, synthetic construct
  REGION 1..7: note = MIA inhibitor
QQLSPLP

SEQ ID NO: 10 - protein, 7 aa, synthetic construct
  REGION 1..7: note = MIA inhibitor
GPPPSPV

SEQ ID NO: 11 - protein, 7 aa, synthetic construct
  REGION 1..7: note = MIA inhibitor
LPLTPLP

SEQ ID NO: 12 - protein, 12 aa, synthetic construct
  REGION 1..12: note = MIA inhibitor
QLNVNHQARADQ

SEQ ID NO: 13 - protein, 12 aa, synthetic construct
  REGION 1..12: note = MIA inhibitor
TSASTRPELHYP

SEQ ID NO: 14 - protein, 12 aa, synthetic construct
  REGION 1..12: note = MIA inhibitor
TFLPHQMHPWPP

SEQ ID NO: 15 - protein, 12 aa, synthetic construct
  REGION 1..12: note = MIA inhibitor
VPHIPPNSMALT

SEQ ID NO: 16 - protein, 12 aa, synthetic construct
  REGION 1..12: note = MIA inhibitor
RLTLLVLIMPAP

SEQ ID NO: 17 - protein, 12 aa, synthetic construct
  REGION 1..12: note = MIA inhibitor
YNLPKVSSNLSP

SEQ ID NO: 18 - protein, 12 aa, synthetic construct
  REGION 1..12: note = MIA inhibitor
MPPTQVSKFRLI

SEQ ID NO: 19 - protein, 12 aa, synthetic construct
  REGION 1..12: note = MIA inhibitor
ANIDATPLFLRA

SEQ ID NO: 20 - protein, 12 aa, synthetic construct
  REGION 1..12: note = MIA inhibitor
LLRTTETLPMFL

SEQ ID NO: 21 - protein, 7 aa, synthetic construct
  REGION 1..7: note = MIA inhibitor
SALEPLV

SEQ ID NO: 22 - protein, 7 aa, synthetic construct
  REGION 1..7: note = MIA inhibitor
GSPTPNA

SEQ ID NO: 23 - protein, 7 aa, synthetic construct
  REGION 1..7: note = MIA inhibitor
APSHATH

SEQ ID NO: 24 - protein, 7 aa, synthetic construct
  REGION 1..7: note = MIA inhibitor
TTVGHSD

SEQ ID NO: 25 - protein, 7 aa, synthetic construct
  REGION 1..7: note = MIA inhibitor
THFSTFT

SEQ ID NO: 26 - protein, 7 aa, synthetic construct
  REGION 1..7: note = MIA inhibitor
SLLLDTS

SEQ ID NO: 27 - protein, 12 aa, synthetic construct
  REGION 1..12: note = MIA inhibitor
SVAMKAHKPLLP

SEQ ID NO: 28 - protein, 12 aa, synthetic construct
  REGION 1..12: note = MIA inhibitor
NTIPGFASKSLD

SEQ ID NO: 29 - protein, 12 aa, synthetic construct
  REGION 1..12: note = MIA inhibitor
VSNYKFYSTTSS

SEQ ID NO: 30 - protein, 12 aa, synthetic construct
  REGION 1..12: note = MIA inhibitor
VSRHQSWHPHDL

SEQ ID NO: 31 - protein, 12 aa, synthetic construct
  REGION 1..12: note = MIA inhibitor
HLNILSTLWKYR

SEQ ID NO: 32 - protein, 12 aa, synthetic construct
  REGION 1..12: note = MIA inhibitor
HNASPSWGSPVM

SEQ ID NO: 33 - protein, 12 aa, synthetic construct
  REGION 1..12: note = MIA inhibitor
SHPWNAQRELSV

SEQ ID NO: 34 - protein, 12 aa, synthetic construct
  REGION 1..12: note = MIA inhibitor
HHWPFWRTLPLS

SEQ ID NO: 35 - protein, 12 aa, synthetic construct
  REGION 1..12: note = MIA inhibitor
WHTKFLPRYLPS

SEQ ID NO: 36 - protein, 12 aa, synthetic construct
  REGION 1..12: note = MIA inhibitor
NNTSFTVVPSVP

SEQ ID NO: 37 - protein, 12 aa, synthetic construct
  REGION 1..12: note = MIA inhibitor
SHLSTWKWWQNR

SEQ ID NO: 38 - protein, 12 aa, synthetic construct
  REGION 1..12: note = MIA inhibitor
FHWHPRLWPLPS

SEQ ID NO: 39 - protein, 12 aa, synthetic construct
  REGION 1..12: note = MIA inhibitor
WHWTYGWRPPAM

SEQ ID NO: 40 - protein, 12 aa, synthetic construct
  REGION 1..12: note = MIA inhibitor
FHWRYPLPLPGQ

SEQ ID NO: 41 - protein, 12 aa, synthetic construct
  REGION 1..12: note = MIA inhibitor
WHWPLFIPNTTA

SEQ ID NO: 42 - protein, 12 aa, synthetic construct
  REGION 1..12: note = MIA inhibitor
WHNGIWWHYGVR

SEQ ID NO: 43 - protein, 12 aa, synthetic construct
  REGION 1..12: note = MIA inhibitor
HHLNYLWPWTRV

SEQ ID NO: 44 - protein, 12 aa, synthetic construct
  REGION 1..12: note = MIA inhibitor
FWHRWSTFPEQP

SEQ ID NO: 45 - protein, 12 aa, synthetic construct
  REGION 1..12: note = MIA inhibitor
WHMSYFWTRPPQ

SEQ ID NO: 46 - protein, 12 aa, synthetic construct
  REGION 1..12: note = MIA inhibitor
FHLNWPSRADYL

SEQ ID NO: 47 - protein, 12 aa, synthetic construct
  REGION 1..12: note = MIA inhibitor
WHKNTNWPWRTL

SEQ ID NO: 48 - protein, 12 aa, synthetic construct
  REGION 1..12: note = MIA inhibitor
ALSPSQSHPVRS

SEQ ID NO: 49 - protein, 12 aa, synthetic construct
  REGION 1..12: note = MIA inhibitor
GTQSTAIPAPTD

SEQ ID NO: 50 - protein, 6 aa, synthetic construct
  REGION 1..6: note = The first residue is Nle, and the fourth is D-Phe
LAHFRW

SEQ ID NO: 51 - protein, 4 aa, synthetic construct
  REGION 1..4: note = The second residue is D-Phe
HFRW